(19) United States
(12) Patent Application Publication
(10) Pub. No.: US 2020/0355936 A1
(43) Pub. Date: Nov. 12, 2020

(54) ESSENTIAL OIL EYEGLASSES

(71) Applicant: Carlos De La Fuente, San Diego, CA (US)

(72) Inventor: Carlos De La Fuente, San Diego, CA (US)

(21) Appl. No.: 16/404,587

(22) Filed: May 6, 2019

(51) Int. Cl. G02C 5/00; A61M 21/02; G02C 11/00

(52) U.S. Cl. CPC G02C 5/001; A61M 2021/0016; G02C 11/00; A61M 21/02

(57) ABSTRACT

Essential oil eyeglasses comprise an eyeglass frame; lenses carried by the frame; and one or more essential oil container assemblies carried by the frame.

### FIELD OF THE INVENTION

The present invention relates to a specialized set of eye glasses that hold essential oils.

### BACKGROUND OF THE INVENTION

Essential oils are highly concentrated fragrant essences extracted from herbs, flowers, fruits, and other plants that give them their unique aromas. They are commonly used in perfume, food flavoring, medicine, massage therapy, and aromatherapy to support a general feeling of well-being. Essential oils also “have a potential curative potential on the body, mind and spirit.

Essential oils come from the process of hydro-distillation, steam distillation, solvent extraction, extraction under pressure, or other mechanical means of extracting oils from various parts of a plant, such as the roots, leaves, seeds, peels, bark, or blossoms. The concentrated liquid extracts contain naturally occurring chemical compounds, including terpenes, esters, aldehydes, ketones, alcohols, phenols, and oxides, which are volatile, meaning they'll evaporate quickly when exposed to air. The different chemical compositions affect the aroma of each essential oil and how it is absorbed and used by the body.

According to the U.S. National Library of Medicine, essential oils are highly concentrated and contain active ingredients. For instance, it takes 220 lbs of lavender flowers to make 1 lb of lavender essential oil. Depending on their use, most essential oils should be diluted first before use. Diluting essential oils helps spread the aromatic, concentrated molecules over a larger area, making it easier and more comfortable for the body to absorb.

Essential oils are most commonly either inhaled as a fine mist of vapor through aromatherapy, or diluted with water or carrier oil, such as almond oil, avocado oil, coconut oil, jojoba oil, or olive oil and applied directly to the skin. When one uses aromatherapy type of vaporizers they are plugged in at home or the office, which means one must be near them to get the benefit. Essential oil fog machines emit essential oil vapors over a large space/range, but a downside to these machines is that there may people in this large space/range that do not like the smell of the essential oil.

There have been many human clinical studies testing the effects of essential oils on healthy subjects. The potential health benefits of essential oils are vast. Many essential oils have antioxidant properties. The results of six studies examining the effects of aromatherapy on depression showed positive effects. Other studies also suggest that essential oils, such as tea tree, lavender, cinnamon, basil or rosemary, may resist microbes and inhibit the growth of strains of E. coli—another promising area of research.

According to a scholarly journal article published in the American Journal of Nursing Science, research has shown that “aromatherapy causes various actions favorable for patients such as relaxation, reductions in anxiety, depression and fatigue, and improvements in quality of life via nervous, endocrine, immune, and circulatory systems, and, therefore, could be applied as a complementary therapy for people with anxiety symptoms. However, as with all complementary treatments, it should not overlap the doctor's instructions, especially in severe cases.

These results may be attributed to phytonutrients, the unique chemical compounds that make up essential oils, which may have a range of health benefits. After all, in nature, these compounds help plants to protect themselves from disease, pests, harsh climate changes and other environmental threats.

### SUMMARY OF THE INVENTION

An aspect of the present invention involves a specialized set of eyeglasses that hold essential oils, which can aid/help with one or more of the following specific uses: relaxation, good sleep, good mood, cleanse toxins from body, higher spirits and pay more attention to work effectively, stimulate lymphatic drainage, rejuvenate energy, purify skin, fight bacteria and fungi, improve immunity, anti-worms, anti-oxidation protection, make people feeling young, relieve skin sensitivity, relieve muscle pain, and/or improve respiratory system. Instead of having to go near the source of essential oil vapors, the specialized set of eyeglasses allows the person wearing them to take the benefits with them. The essential oil vapors are only in a small range adjacent the user's nose/head.

Another aspect of the invention involves essential oil eyeglasses comprising an eyeglass frame; lenses carried by the frame; and one or more essential oil container assemblies carried by the frame.

One or more implementations of the immediately above aspect of the invention involves one or more of the following: the one or more essential oil container assemblies include a sponge and an essential oil absorbed by the sponge; the one or more essential oil container assemblies include an outer wall that faces laterally outward from the eyeglass frame, away from a user wearing the essential oil eyeglasses, and an inner wall that faces laterally inward from the eyeglass frame, towards the user wearing the essential oil eyeglasses; at least one of the outer wall and the inner wall include a set of air holes that allow essential oil gases to pass there through; the one or more essential oil container assemblies include an inner wall, a front wall, a rear wall, a bottom wall, an open top, and a cap; a container receiving assembly and the front wall includes a concave latch and the container receiving assembly includes a convex latch that is operably associated with the concave latch to detachably couple the one or more essential oil container assemblies within the container receiving assembly; the eyeglass frame includes temples having one or more additional sets of air holes separate from the set of air holes, the one or more additional sets of air holes communicating with the one or more essential oil container assemblies for emitting essential oil fragrances in additional areas of the essential oil eyeglasses; the one or more essential oil container assemblies include a removable cap that is removable and attachable with respect to the one or more essential oil container assemblies to add essential oil; the eyeglass frame includes end pieces and temples pivotally coupled to the end pieces, the one or more essential oil container assemblies disposed in the end pieces; and/or the lenses are at least one of blue light block lenses and computer screen lenses.

These and other aspects and advantages of the present invention will become apparent from the following detailed description, which, when taken in conjunction with the accompanying drawings, illustrates by way of example the principles of the invention.

### BRIEF DESCRIPTION OF DRAWINGS

The preferred embodiments of the invention will hereinafter be described in conjunction with the appended drawings provided to illustrate and not to limit the invention, in which:

FIG. 1 is a front perspective view of an embodiment of a specialized set of eyeglasses that hold essential oils.

FIG. 2 is a side perspective view of the specialized set of eyeglasses that hold essential oils of FIG. 1.

FIG. 3 is an enlarged perspective view of an embodiment of a container for the specialized set of eyeglasses that hold essential oils of FIGS. 1 and 2.

FIG. 4 is an exploded perspective view of the container for the specialized set of eyeglasses that hold essential oils of FIGS. 1 and 2.

FIG. 5 is a perspective view of another embodiment of a container for a specialized set of eyeglasses that hold essential oils.

While the various features of this invention are hereinafter described and illustrated as being particularly adapted for providing rigidity to instrument devices the invention is not limited to the embodiments illustrated in the drawings but are merely used to illustrate the wide variety of uses of this invention. Therefore, the foregoing is considered as illustrative only of the principles of the invention. Since numerous modification and changes will readily occur by those skilled in the art, it is not desired to limit the invention to the exact construction and operation shown and described and accordingly, all suitable modifications and equivalents may be resorted to, falling within the scope of the invention.

### DESCRIPTION OF EMBODIMENT OF THE INVENTION

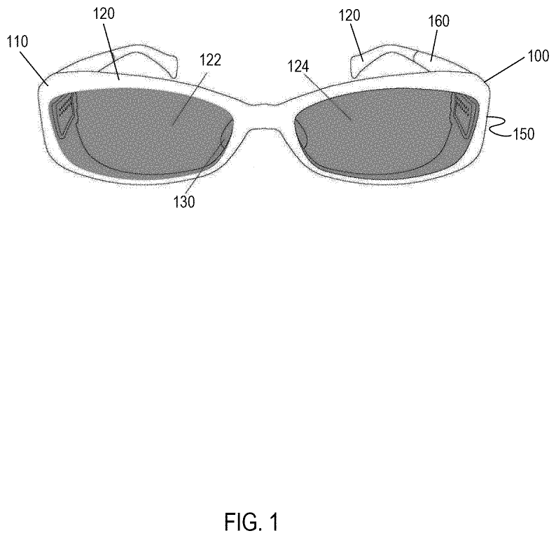

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

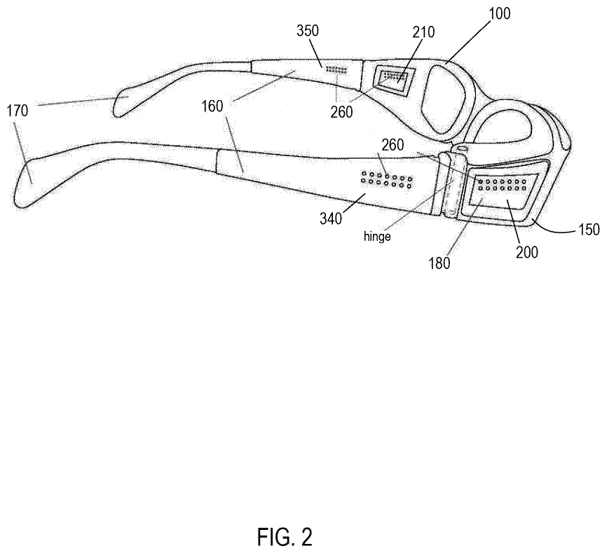

With respect to FIGS. 1-4, an embodiment of a specialized set of eyeglasses 100 that hold essential oils will be described. The eyeglasses 100 include an eyeglass frame 110 with rims 120 that hold blue light block/computer screen lenses 122, 124, nose bridge 130, nose pad 140, end pieces 150, and temples/arms 152 pivotally connected to the end pieces 150 at hinges 160 and terminating at temple tips 170.

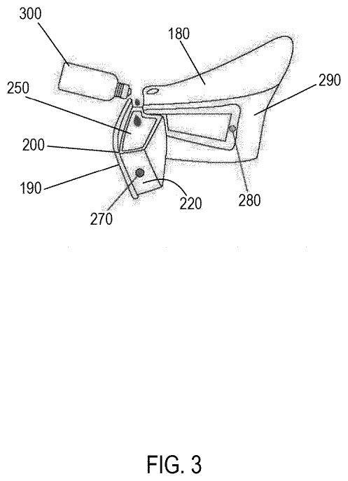

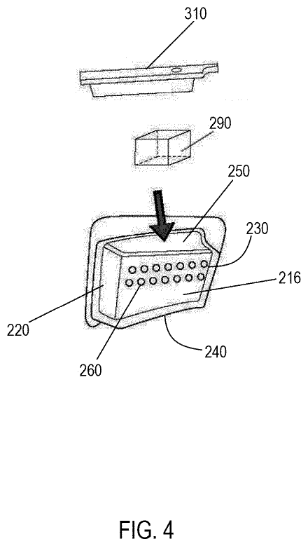

With reference additionally to FIGS. 3 and 4, an embodiment of an essential oil container assembly 180 is shown. The container assembly 180 includes a container 190 with an outer wall 200 that faces laterally outward from the end pieces 150, an inner wall 210 that faces laterally inward from the end pieces 150, a front wall 220, a rear wall 230, a bottom wall 240, and an open top 250. The outer wall 200 and the inner wall 210 include air holes 260 that allow the essential oil gases to pass there through. The front wall 220 includes a concave latch 270 and that is operably associated with a convex latch 280 of a container receiving assembly 290 of the end pieces 150. The essential oil container assembly(ies) 380 are pivotally connected to the container receiving assembly(ies) 290 and laterally insertable into the end pieces 150. The container 190 holds a sponge 290, which receives essential oils from an essential oil fluid source 300. A cap 310 is removably attachable to the container 190 to cover the open top 250 of the container 190. Temples/arms 152 include end portions 320 with air holes 260 on both outer and inner sides 340, 350 that communicate with the essential oil container assembly 180 for emitting essential oil fragrances in these additional areas.

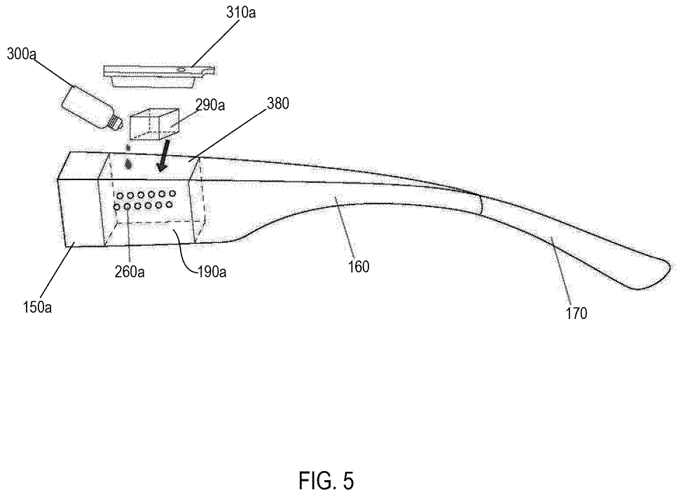

With reference to FIG. 5, another embodiment of an essential oil container assembly 380 is shown. Elements of the essential oil container assembly 380 and eyeglasses that are similar to the elements of the essential oil container assembly 180 shown and described herein are shown with like reference numbers, but with an “a” suffix. The prior description of these similar elements is incorporated herein. Instead of the essential oil container assembly(ies) 380 being pivotally connected to the container receiving assembly 290 and laterally insertable into the end pieces 150 like the essential oil container assembly 180, the essential oil container assembly(ies) 380 is/are disposed in the end pieces 150a and accessible by removably attachable cap 310a that is removable/attachable from above the container 190a for adding essential oils from the essential oil fluid source 300a to the sponge 290a. Air holes 260a allow the essential oil gases to pass there through.

The specialized set of eyeglasses 100 hold essential oils, which can aid/help with one or more of the following specific uses: relaxation, good sleep, good mood, cleanse toxins from body, higher spirits and pay more attention to work effectively, stimulate lymphatic drainage, rejuvenate energy, purify skin, fight bacteria and fungi, improve immunity, anti- worms, anti-oxidation protection, make people feeling young, relieve skin sensitivity, relieve muscle pain, and/or improve respiratory system. Instead of having to go near the source of essential oil vapors, the specialized set of eyeglasses 100 allow the person wearing them to take the benefits with them. The essential oil vapors are only in a small range adjacent the user's nose/head.

The above figures may depict exemplary configurations for the invention, which is done to aid in understanding the features and functionality that can be included in the invention. The invention is not restricted to the illustrated architectures or configurations but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus, the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

I claim:

1. Essential oil eyeglasses, comprising:
eyeglass frame;
lenses carried by the frame; and
one or more essential oil container assemblies carried by the frame.

2. The essential oil eyeglasses of claim 1, wherein the one or more essential oil container assemblies include a sponge and an essential oil absorbed by the sponge.

3. The essential oil eyeglasses of claim 1, wherein the one or more essential oil container assemblies include an outer wall that faces laterally outward from the eyeglass frame, away from a user wearing the essential oil eyeglasses, and an inner wall that faces laterally inward from the eyeglass frame, towards the user wearing the essential oil eyeglasses.

4. The essential oil eyeglasses of claim 3, wherein at least one of the outer wall and the inner wall include a set of air holes that allow essential oil gases to pass there through.

5. The essential oil eyeglasses of claim 1, wherein the one or more essential oil container assemblies include an inner wall, a front wall, a rear wall, a bottom wall, an open top, and a cap.

6. The essential oil eyeglasses of claim 5, further including a container receiving assembly and the front wall includes a concave latch and the container receiving assembly includes a convex latch that is operably associated with the concave latch to detachably couple the one or more essential oil container assemblies within the container receiving assembly.

7. The essential oil eyeglasses of claim 1, wherein the eyeglass frame includes temples having one or more additional sets of air holes separate from the set of air holes, the one or more additional sets of air holes communicating with the one or more essential oil container assemblies for emitting essential oil fragrances in additional areas of the essential oil eyeglasses.

8. The essential oil eyeglasses of claim 1, wherein the one or more essential oil container assemblies include a removable cap that is removable and attachable with respect to the one or more essential oil container assemblies to add essential oil.

9. The essential oil eyeglasses of claim 1, wherein the eyeglass frame includes end pieces and temples pivotally coupled to the end pieces, the one or more essential oil container assemblies disposed in the end pieces.

10. The essential oil eyeglasses of claim 1, wherein the lenses are at least one of blue light block lenses and computer screen lenses.
